(19) United States
(12) Patent Application Publication
(10) Pub. No.: US 2024/0335659 A1
(43) Pub. Date: Oct. 10, 2024

(54) APPARATUS AND METHOD FOR APPLYING MONOPOLAR AND BIPOLAR CURRENTS BY SKIN DEPTH DURING NEEDLE INSERTION

(71) Applicants: Suk Bae SEO, Seoul (KR); Ja Young KIM, Seoul (KR); Ho Joon SEO, Seoul (KR)

(72) Inventors: Suk Bae SEO, Seoul (KR); Ja Young KIM, Seoul (KR); Ho Joon SEO, Seoul (KR)

(21) Appl. No.: 18/750,380

(22) Filed: Jun. 21, 2024

Related U.S. Application Data
(63) Continuation of application No. PCT/KR2022/021027, filed on Dec. 22, 2022.

(30) Foreign Application Priority Data
Dec. 22, 2021 (KR) 10-2021-0184517
Jul. 1, 2022 (KR) 10-2022-0081421

(51) Int. Cl. A61N 1/32; A61N 1/04; A61N 1/06; A61N 1/36

(52) U.S. Cl. CPC A61N 1/328; A61N 1/0476; A61N 1/06; A61N 1/36017; A61N 1/36034

(57) ABSTRACT

Disclosed is accurately apply abundant or efficient electric energy during high-frequency irradiation.

### CROSS-REFERENCE TO RELATED APPLICATIONS

The present application is a continuation of International Patent Application No. PCT/KR2022/021027, filed on Dec. 22, 2022, which is based upon and claims the benefit of priority to Korean Patent Application Nos. 10-2021-0184517 filed on Dec. 22, 2021 and 10-2022-0081421 filed on Jul. 1, 2022. The disclosures of the above-listed applications are hereby incorporated by reference herein in their entirety.

### BACKGROUND

The present disclosure relates to an apparatus and a method for applying monopolar and bipolar currents. More specifically, the present disclosure relates to an apparatus and a method for applying monopolar and bipolar currents by skin depth during needle insertion.

In general, skin care devices for wrinkle removal, skin elasticity restoration, and sebum removal include a method of transmitting ultrasound to skin tissue (HIFU type), a method of transmitting high frequency waves to skin tissue (RF type), and a method of irradiating laser light to skin tissue (Optical type).

A device employing the method of transmitting high frequency to skin tissue repeatedly infiltrates the deep portion (e.g., the dermal layer) of the skin with RF needle electrodes that reciprocate in a vertical direction, and removes damaged collagen and elastic fibers from the deep portion of the skin of the target area and promote new formation, using heat generated by high frequencies.

Furthermore, these skin care devices improve skin pigmentation, acne marks, and wrinkles.

In other words, after intentionally causing a wound by applying various energies to the target area of the skin, the skin area is regenerated by stimulating the collagen in the dermal layer and inducing collagen regeneration.

However, conventional skin care devices were unable to deliver abundant or efficient electrical energy to a specific depth of the skin.

Accordingly, conventional skin care devices cannot accurately irradiate abundant or efficient electric energy when performing high-frequency irradiation, and the accuracy of high-frequency irradiation has been reduced.

In addition, conventional skin care devices have limitations in shortening the high-frequency irradiation time and maximizing the effect of high-frequency irradiation because the doctor needs to carefully perform the high-frequency irradiation.

### PRIOR ART LITERATURE

### Patent Literature

(Patent Document 1) Korean registered patent KR 10-0985822 (announced on Oct. 8, 2010)

### SUMMARY

Embodiments of the present disclosure provide an apparatus and a method capable of increasing the accuracy of high-frequency irradiation by accurately irradiating abundant electric energy or efficient electric energy during high-frequency irradiation.

Further, embodiments of the present disclosure provide an apparatus and a method capable of shortening a high-frequency irradiation time and maximizing high-frequency irradiation effects.

Further, embodiments of the present disclosure provide an apparatus and a method capable of maximizing optimal skin improvement effects of a monopolar type and a bipolar type while preventing skin burns in advance.

However, problems to be solved by the present disclosure may not be limited to the above-described problems. Although not described herein, other problems to be solved by the inventive concept can be clearly understood by those skilled in the art from the following description.

According to an embodiment, an apparatus for applying monopolar and bipolar currents by skin depth during needle insertion includes an electrode part including a plurality of needle-type electrodes inserted into skin, an energy supply part that selectively applies a monopolar first current and a bipolar second current to the plurality of electrodes, a transfer part that moves the electrode part such that the plurality of electrodes reach a target depth in the skin, and a processor that controls the energy supply part such that the first current and the second current are applied to the plurality of electrodes at a current intensity corresponding to the reached target depth when the plurality of electrodes has reached the target depth, and the processor may perform control such that the first current and the second current are sequentially applied to the plurality of electrodes at a current intensity corresponding to a present first depth from a moment the electrode part is inserted into the skin, when the target depth is the first depth, and perform control such that the second current and the first current are sequentially applied to the plurality of electrodes at a current intensity corresponding to a preset second depth from the moment the electrode part is inserted into the skin when the target depth is the second depth.

The processor may perform control such that the first current and the second current are sequentially applied to the plurality of electrodes at the current intensity corresponding to the first depth until the electrode part is pulled out from the moment the electrode part is inserted into the skin when the target depth is the preset first depth.

The processor may perform control such that there is a predetermined rest period between application times of the first current and the second current.

The processor may perform control such that the second current and the first current are sequentially applied to the plurality of electrodes at the current intensity corresponding to the second depth until the electrode part is pulled out from the moment the electrode part is inserted into the skin when the target depth is the preset second depth.

The processor may perform control such that there is a predetermined rest period between application times of the second current and the first current.

According to an embodiment, a method for being performed by an apparatus for applying monopolar and bipolar currents by skin depth during needle insertion includes moving, by a transfer part of the apparatus, an electrode part of the apparatus such that a plurality of electrodes reach a target depth in skin, determining, by a processor of the apparatus, whether the plurality of electrodes has reached the target depth, and controlling, by the processor of the apparatus, an energy supply part of the apparatus such that a monopolar first current and a bipolar second current are applied to the plurality of electrodes at a current intensity corresponding to the reached target depth when the plurality of electrodes has reached the target depth, and the controlling includes performing control such that the first current and the second current are sequentially applied to the plurality of electrodes at a current intensity corresponding to a first depth from the moment the electrode part is inserted into the skin, when the target depth is the preset first depth, and performing control such that the first current and the second current are sequentially applied to the plurality of electrodes at a current intensity corresponding to a second depth from the moment the electrode part is inserted into the skin, when the target depth is the preset second depth.

The controlling may include performing control such that the first current and the second current are sequentially applied to the plurality of electrodes at the current intensity corresponding to the first depth until the electrode part is pulled out from the moment the electrode part is inserted into the skin when the target depth is the preset first depth.

The controlling may include performing control such that there is a predetermined rest period between application times of the first current and the second current.

The controlling may include performing control such that the second current and the first current are sequentially applied to the plurality of electrodes at the current intensity corresponding to the second depth until the electrode part is pulled out from the moment the electrode part is inserted into the skin when the target depth is the preset second depth.

The controlling may include performing control such that there is a predetermined rest period between application times of the second current and the first current.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram showing a configuration of an apparatus for applying monopolar and bipolar currents by skin depth during needle insertion according to the present disclosure;

FIG. 2 is a diagram showing an example of a process in which a plurality of needle-type electrodes of FIG. 1 invades the inside of skin;

FIG. 3 is a diagram showing the structure of a plurality of needle-type electrodes disposed in the electrode part of FIG. 1;

FIG. 4 is a diagram showing an example of a process of applying a monopolar type first current to a plurality of needle-type electrodes of FIG. 3;

FIG. 5 is a diagram showing an example of a process of applying a bipolar type second current to a plurality of needle-type electrodes in FIG. 3;

FIG. 6 is a flowchart showing a method of applying monopolar and bipolar currents by skin depth during needle insertion according to the present disclosure;

FIGS. 7 and 8 are diagrams showing a comparison of the intensity of the monopolar type first current and the intensity of the bipolar type second current until the plurality of electrodes of FIG. 2 has reached a target depth in a dermal layer;

FIGS. 9 to 12 are diagrams showing an example of a process of performing control such that a first current and a second current are sequentially applied to a plurality of electrodes in the control step of FIG. 6; and

FIGS. 13 to 16 are diagrams illustrating an example of a process of performing control such that a second current and a first current are sequentially applied to a plurality of electrodes in the control step of FIG. 6.

### DETAILED DESCRIPTION

Like numerals refer to like elements throughout the specification. Not all elements of embodiments of the disclosure will be described, and description of what are commonly known in the art or what overlap each other in the embodiments of the disclosure will be omitted. The term ‘part, module, member, or block’ used in the specification may be implemented as software or hardware, and depending on the embodiment, a plurality of ‘parts, modules, members, or blocks’ may be implemented as a single component, or it is also possible for one ‘part, module, member, or block’ to include multiple components.

It will be further understood that the term “connect” or its derivatives refer both to direct and indirect connection, and the indirect connection includes a connection over a wireless communication network.

The term “include (or including)” or “comprise (or comprising)” is inclusive or open-ended and does not exclude additional, unrecited elements or method steps, unless otherwise mentioned.

Further, when it is stated that a layer is “on” another layer or substrate, the layer may be directly on another layer or substrate or a third layer may be disposed therebetween.

Terms such as first and second are used to distinguish one component from another component, and the components are not limited by the above-mentioned terms.

As used herein, singular forms may include plural forms as well unless the context clearly indicates otherwise.

In each step, identification symbols are used for convenience of description and the identification symbols do not describe the order of the steps and the steps may be performed in a different order from the described order unless the context clearly indicates a specific order.

Hereinafter, the operating principle and embodiments of the present disclosure will be described with reference to the attached drawings.

An apparatus for applying monopolar and bipolar currents by skin depth during needle insertion, according to the present disclosure, is used to increase the temperature of an electromagnetically-energized target area under the skin surface through a microneedle to a level that causes tissue heating.

In this specification, the control unit of the apparatus for applying monopolar and bipolar currents by skin depth during needle insertion according to the present disclosure includes various devices capable of performing calculation processing and providing results to a user. For example, the control unit of the apparatus for applying monopolar and bipolar currents by skin depth during needle insertion according to the present disclosure may include all of a computer, a server device, and a portable terminal, or may take the form of any one thereof.

Here, the computer may include, for example, a notebook, a desktop, a laptop, a tablet PC, a slate PC, which are equipped with a web browser, and the like.

The server device is a server that processes information by communicating with external devices and may include an application server, computing server, database server, file server, mail server, proxy server, web server and the like.

For example, the portable terminal may be a wireless communication device that is portable and mobile and may include all types of handheld-type wireless communication devices, such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), or WiBro (Wireless Broadband Internet) terminals, and wearable devices such as watches, rings, bracelets, anklets, necklaces, glasses, contact lenses, or head-mounted-device (HMD), or the like.

The apparatus for applying monopolar and bipolar currents by skin depth during needle insertion according to the present disclosure may control an energy supply part that selectively applies a monopolar first current and a bipolar second current to a plurality of needle-type electrodes such that, when a target depth is reached by the plurality of needle-type electrodes inserted into the skin, the monopolar first current and the bipolar second current are alternately applied to the plurality of electrodes at a current intensity corresponding to the target depth reached. In this case, the intensities of the first current and the second current increases as the target depth increases, but the intensities may be different from each other.

The apparatus for applying monopolar and bipolar currents by skin depth during needle insertion may accurately apply abundant or efficient electric energy during high-frequency irradiation, thereby increasing the accuracy of high-frequency irradiation. In addition, the apparatus for applying monopolar and bipolar currents by skin depth during needle insertion may shorten a high-frequency irradiation time and maximize the effect of high-frequency irradiation. In addition, the apparatus for applying monopolar and bipolar currents by skin depth during needle insertion may achieve the optimal skin condition of the monopolar type and the bipolar type while preventing skin burns in advance.

Hereinafter, the apparatus for applying monopolar and bipolar currents by skin depth during needle insertion will be described in detail.

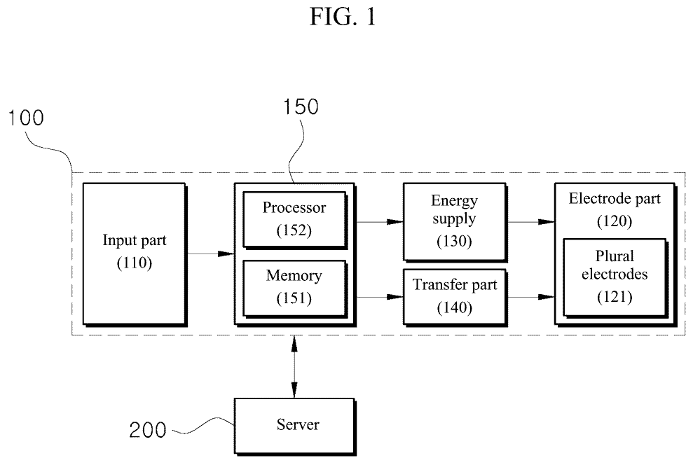

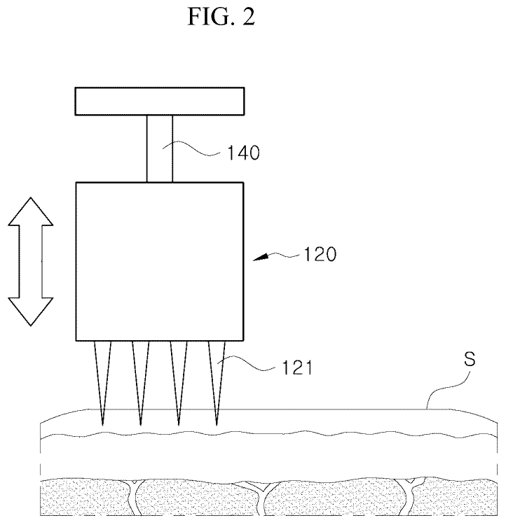

FIG. 1 is a diagram showing a configuration of an apparatus for applying monopolar and bipolar currents by skin depth during needle insertion according to the present disclosure. FIG. 2 is a diagram showing an example of a process in which a plurality of needle-type electrodes of an electrode part invades the inside of skin due to the movement of a transfer part of FIG. 1.

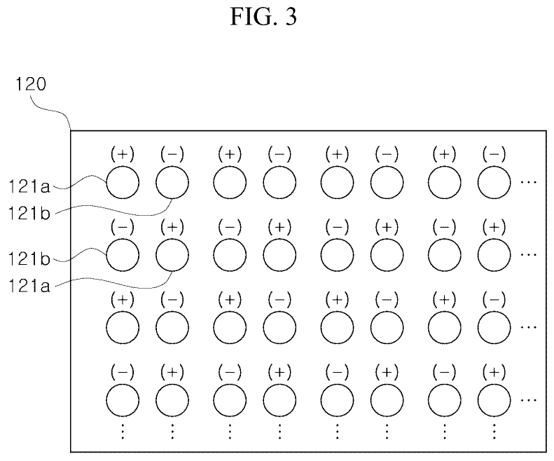

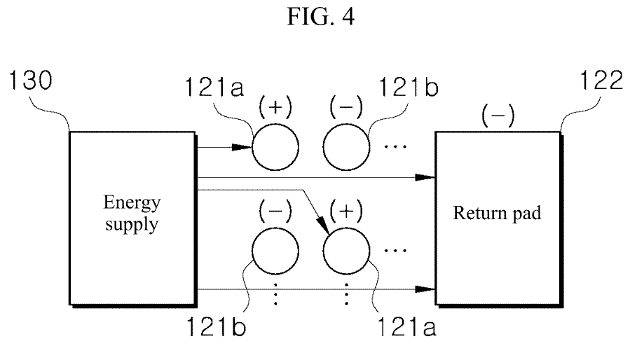

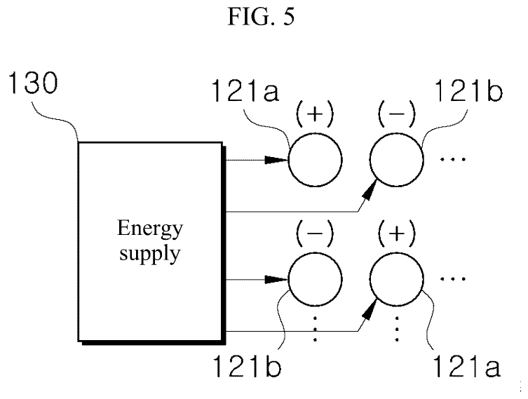

FIG. 3 is a diagram showing the structure of a plurality of needle-type electrodes disposed in the electrode part of FIG. 1. FIG. 4 is a diagram showing an example of a process of applying a monopolar first current to a plurality of needle-type electrodes of FIG. 3. FIG. 5 is a diagram showing an example of a process of applying a bipolar second current to a plurality of needle-type electrodes in FIG. 3.

Referring to FIGS. 1 to 5, an apparatus for applying monopolar and bipolar currents by skin depth during needle insertion 100 may include an input part 110, an electrode part 120, an energy supply part 130, a transfer part 140, and a controller 150.

The input part 110 is for receiving target depth information for the skin from a user, and when the target depth information for the skin is input, the controller 150 may control the operation of the apparatus to correspond to the input target depth information for the skin. Here, the target depth information for the skin may be a target depth value within a dermal layer.

As such, the input part 110 may include hardware physical keys (e.g., a button, dome switch, jog wheel, jog switch, or the like located on at least one of the front, back, and sides of the apparatus) and software touch keys. As an example, the touch key may include a virtual key, a soft key, or a visual key displayed on a touch screen-type display through software processing, or a touch key disposed in the other portion than the touch screen. Meanwhile, the virtual key or visual key may be displayed on the touch screen while having various forms, and may be made of, for example, a graphic, text, icon, video, or a combination thereof.

The electrode part 120 may include a plurality of needle-type electrodes 121 that are inserted into the skin ‘S’. The plurality of needle-type electrodes 121 invade the deeper portion of the skin ‘S’ (e.g., the dermal layer) and use heat generated by a high frequency to remove damaged collagen, elastic fibers, or the like from the deeper portion of the targeted area and promote new formation.

As shown in FIG. 3, a plurality of electrodes 121a of positive (+) polarity and a plurality of electrodes 121b of negative (−) polarity of the plurality of needle-type electrodes 121 may be disposed on the electrode part 120 in a plurality of groups, and the plurality of electrodes 121b of negative (−) polarity and the plurality of electrodes 121a of positive (+) polarity may be disposed in a plurality of groups. For example, the plurality of electrodes 121a with positive (+) polarity and the plurality of electrodes 121b with negative (−) polarity may be repeatedly arranged in the form of a plurality of groups in odd-numbered rows of the electrode part 120, and the plurality of electrodes 121b with negative (−) polarity and the plurality of electrodes 121a with positive (+) polarity may be repeatedly arranged in the form of a plurality of groups in even-numbered rows of the electrode part 120.

The energy supply part 130 may selectively apply the monopolar first current and the bipolar second current to the plurality of electrodes 121. Here, the energy supply part 130 may include a switching circuit for selectively applying the monopolar first current and the bipolar second current.

In this case, as shown in FIG. 4, the energy supply part 130 may apply the monopolar first current to the plurality of electrodes 121a with positive (+) polarity of the plurality of electrodes 121 and a return pad 122 with negative (−) polarity. In order for a high-frequency current to flow through the skin ‘S’, a current circuit needs to be formed. When the energy supply part 130 applies the monopolar first current to the plurality of electrodes 121a with positive (+) polarity and the return pad 122 with negative (−) polarity, high-frequency energy is concentrated on the plurality of electrodes 121a with positive (+) polarity, enabling deeper heat transfer than the bipolar type, and generating abundant deep heat. Here, the return pad 122 may be a pad for grounding.

In addition, as shown in FIG. 5, the energy supply part 130 may apply the bipolar second current to the plurality of electrodes 121a with positive (+) polarity of the plurality of electrodes 121 and the plurality of electrodes 121b with negative (−) polarity. When the energy supply part 130 applies the bipolar second current to the plurality of electrodes 121a with positive (+) polarity and the plurality of electrodes 121b with negative (−) polarity, a current circuit is formed between the plurality of electrodes 121a with positive (+) polarity and the plurality of electrodes 121b with negative (−) polarity, preventing excessive heat transfer and enabling concentration on the selected high-frequency irradiation area.

The transfer part 140 may move the electrode part 120 such that the plurality of electrodes 121 reach the target depth in the skin. The transfer part 140 may move the electrode part in the vertical direction.

The controller 150 may be implemented with a memory 151 that stores an algorithm for controlling the operations of components within the apparatus or data for program reproducing the algorithm, and at least one processor 152 that performs the aforementioned operations using the data stored in the memory 151. Here, the memory 151 and the processor 152 may be implemented as separate chips. Also, the memory 151 and the processor 152 may be implemented as a single chip.

The memory 151 may store data supporting various functions of the host device and programs for operation of the processor, may store input/output data, and may store a number of applications (application programs or applications) running on the host device, and data and instructions for operation of the host device. At least some of these application programs may be downloaded from an external server through wireless communication.

The memory 151 may include at least one type of storage medium among a flash memory type, a hard disk type, a solid state disk (SSD) type, a Silicon Disk Drive type (SDD) type, a multimedia card micro type, a card type memory (e.g., SD or XD memory), random access memory (RAM), static random access memory (SRAM), read-only memory (ROM), electrically erasable programmable read-only memory (EEPROM) programmable read-only memory (PROM), magnetic memory, magnetic disk, and optical disk. Furthermore, the memory 151 may be a database that is separate from the host device but connected thereto in a wired or wireless manner.

When the plurality of electrodes 121 reach the target depth of the skin layer, the processor 152 may control the energy supply part 130 such that the monopolar first current and the bipolar second current are applied to the plurality of electrodes 121 at a current intensity corresponding to the reached target depth. In this case, the intensities of the monopolar first current and the bipolar second current increase as the target depth increases, and the intensities may be different from each other. For example, as the target depth becomes deeper, the intensity of the monopolar first current may be larger or smaller than the intensity of the bipolar second current.

Meanwhile, the processor 152 may control a display device to monitor the plurality of electrodes 121 inserted according to the insertion depth each time the plurality of electrodes 121 are inserted into the skin layer.

In addition, the processor 152 may measure an impedance for each current for each insertion depth each time the plurality of electrodes 121 are inserted into the skin layer, and adjust the energy for each insertion depth based on the measured impedance for calibration.

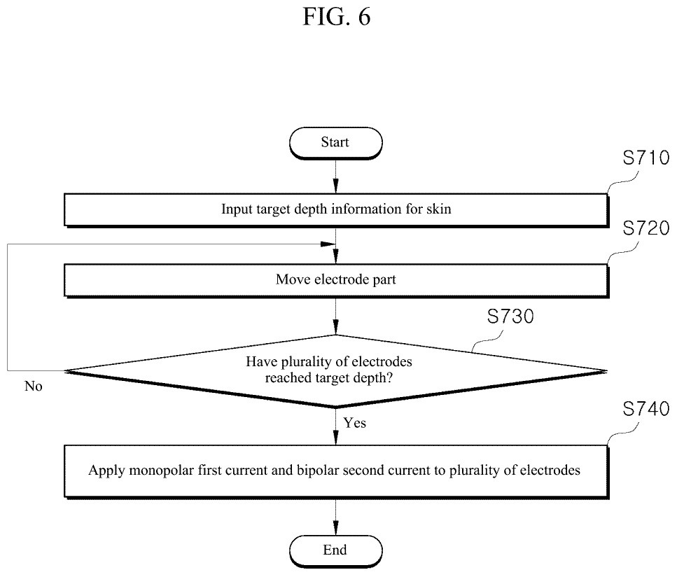

FIG. 6 is a flowchart showing a method of applying monopolar and bipolar currents by skin depth during needle insertion according to the present disclosure.

Referring to FIG. 6, the method of applying monopolar and bipolar currents by skin depth during needle insertion may include an input step (S710), a transfer step (S720), a determination step (S730), and a control step (S740).

In the input step, a target depth in the skin may be input from a user through the input part 110 (S710). Here, the target depth information for skin may be a target depth value within the dermal layer.

In the transfer step, the transfer part 140 may move the electrode part 120 such that the plurality of electrodes 121 reach the target depth within the skin ‘S’ (S720).

In the determination step, the processor 152 may determine whether the plurality of electrodes 121 has reached the target depth (S730).

In the control step, when the plurality of electrodes 121 has reached the target depth, the processor 152 may control the energy supply part 130 such that the monopolar first current and the bipolar second current are applied to the plurality of electrodes 121 at a current intensity corresponding to the reached target depth (S740). The energy supply part 130 may sequentially apply the monopolar first current and the bipolar second current to the plurality of electrodes 121.

In this case, the intensities of the monopolar first current and the bipolar second current increase as the target depth increases, and the intensities may be different from each other. For example, as the target depth becomes deeper, the intensity of the monopolar first current may be larger than the intensity of the bipolar second current. For another example, as the target depth becomes deeper, the intensity of the bipolar second current may be greater than the intensity of the monopolar first current.

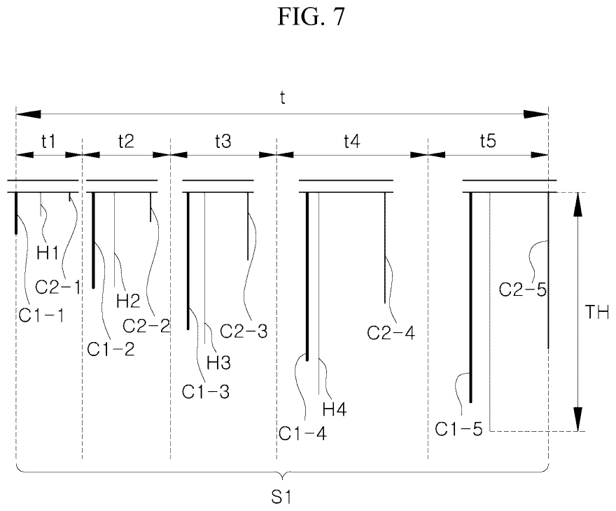

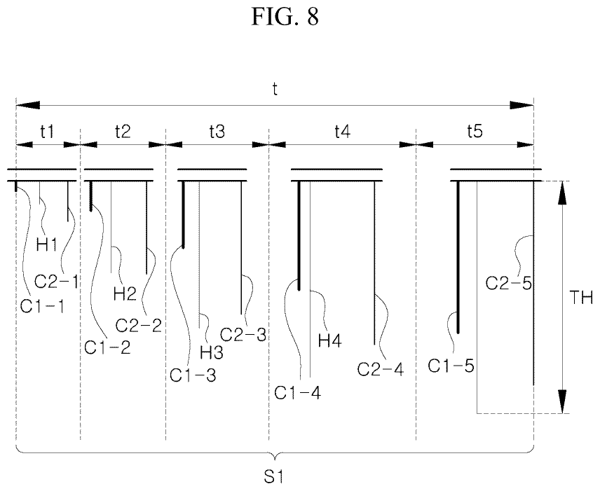

FIGS. 7 and 8 are diagrams showing a comparison of the intensity of the monopolar first current and the intensity of the bipolar second current until the plurality of electrodes of FIG. 2 has reached a target depth in a dermal layer.

As shown in FIG. 7, during the first to fifth time periods t1 to t5 of the total time period (t) for which the plurality of electrodes 121 has reached target depths H1 to TH in the dermal layer S1, intensities C1-1 to C1-5 of the monopolar first current may be greater than intensities C2-1 to C2-5 of the bipolar second current. In addition, as shown in FIG. 8, during the first to fifth time periods t1 to t5 of the total time period (t) for which the plurality of electrodes 121 has reached target depths H1 to TH in the dermal layer S1, the intensities C2-1 to C2-5 of the bipolar second current may be greater than the intensities C1-1 to C1-5 of the monopolar first current.

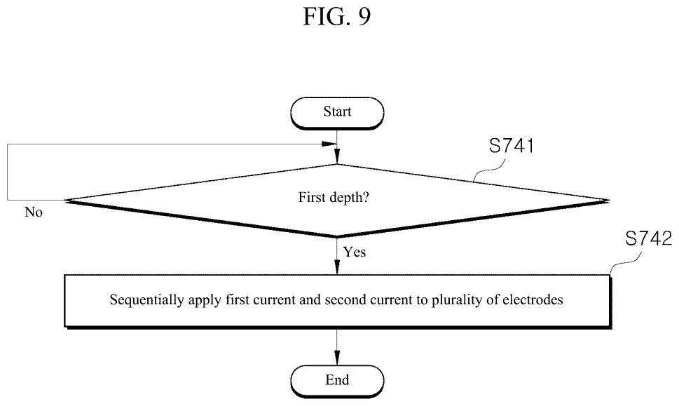

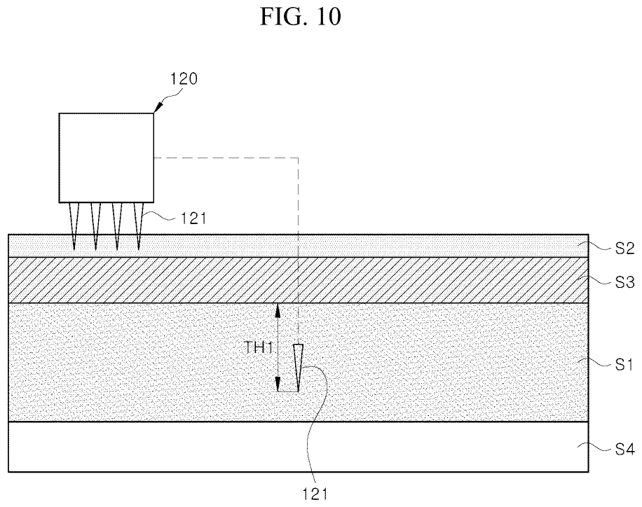

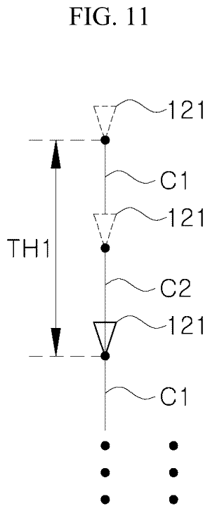

FIGS. 9 to 12 are diagrams showing an example of a process of performing control such that a first current and a second current are sequentially applied to a plurality of electrodes in the control step of FIG. 6.

Referring to FIGS. 9 to 11, the control step may determine whether the target depth is a preset first depth TH1 through the processor 152 (S741). In the control step, when the target depth in the dermal layer S1 is determined to be the preset first depth TH1, from the moment the plurality of electrodes 121 are inserted into the skin, the energy supply part 130 may be controlled through the processor 152 such that the monopolar first current C1 and the bipolar second current C2 are sequentially applied to the plurality of electrodes 121 at a current intensity corresponding to the first depth TH1 (S740). Furthermore, in the control step, when the target depth in the dermal layer S1 is determined to be the preset first depth THI, until the plurality of electrodes 121 are pulled out of the skin from the moment the plurality of electrodes 121 are inserted into the skin, the energy supply part 130 may be controlled through the processor 152 such that the monopolar first current C1 and the bipolar second current C2 are sequentially applied to the plurality of electrodes 121 at a current intensity corresponding to the first depth TH1. Herein, S2 may be the stratum corneum, S3 may be the epidermal layer, and S4 may be the subcutaneous tissue layer.

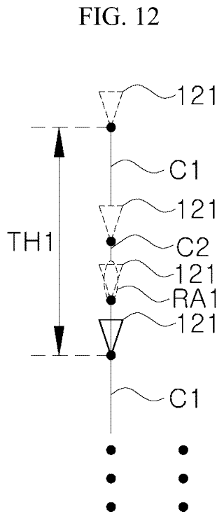

In this case, as shown in FIG. 12, the processor 152 may perform control such that there is a predetermined rest period RA1 between the application times of the bipolar second current C2 and the monopolar first current C1. The rest period RA1 is a time period in which the bipolar second current C2 is not applied for a preset time, and the present disclosure provides the rest period RA1, thus maximizing the optimal skin improvement effect of the bipolar type and the monopolar type while preventing skin burns.

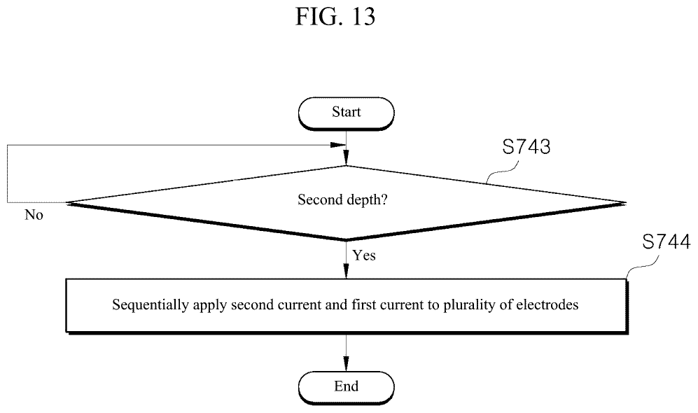

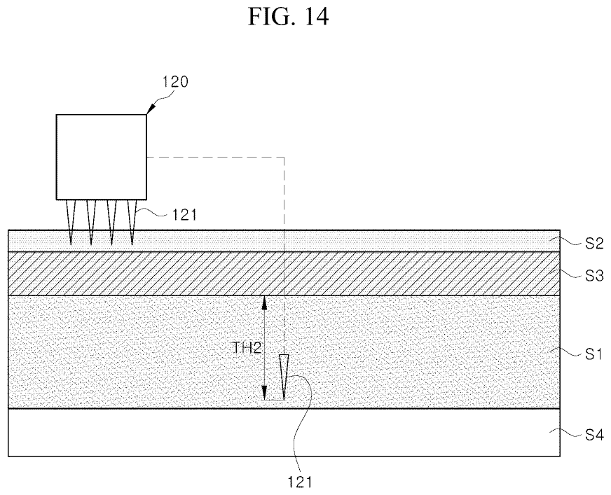

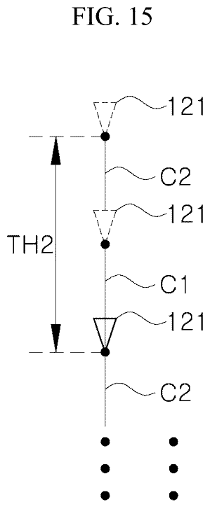

FIGS. 13 to 16 are diagrams illustrating an example of a process of performing control such that a second current and a first current are sequentially applied to a plurality of electrodes in the control step of FIG. 6.

Referring to FIGS. 13 to 15, the control step may determine whether a target depth is a preset second depth TH2 through the processor 152 (S743). In the control step, when the target depth in the dermal layer S1 is determined to be the preset second depth TH2, from the moment the plurality of electrodes 121 are inserted into the skin, the energy supply part 130 may be controlled through the processor 152 such that the bipolar second current C2 and the monopolar first current C1 are sequentially applied to the plurality of electrodes 121 at a current intensity corresponding to the second depth TH2. Furthermore, in the control step, when the target depth in the dermal layer S1 is determined to be the preset second depth TH2, until the plurality of electrodes 121 are pulled out of the skin from the moment the plurality of electrodes 121 are inserted into the skin, the energy supply part 130 may be controlled through the processor 152 such that the bipolar second current C2 and the monopolar first current C1 are sequentially applied to the plurality of electrodes 121 at a current intensity corresponding to the second depth TH2. Herein, S2 may be the stratum corneum, S3 may be the epidermal layer, and S4 may be the subcutaneous tissue layer.

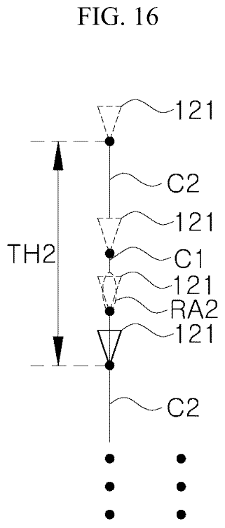

In this case, as shown in FIG. 16, the processor 152 may perform control such that there is a predetermined rest period RA2 between the application times of the monopolar first current C1 and the bipolar second current C2. The rest period RA2 is a time period in which the monopolar first current C1 is not applied for a preset time, and the present disclosure provides the rest period RA2, thus maximizing the optimal skin improvement effect of the monopolar type and the bipolar type while preventing skin burns.

According to the performance of the components illustrated in FIGS. 1 to 5, at least one component may be added or deleted. Additionally, it will be easily understood by those skilled in the art that the mutual positions of the components may be changed in response to the performance or structure of the system.

Although it is described with reference to FIGS. 6, 9, and 13 that a plurality of steps are sequentially performed, this is merely illustrative of the technical idea of the embodiment. Those of ordinary skill in the art to which this embodiment belongs may perform various modifications and variations such as changing the order described in FIGS. 6, 9, and 13 or performing one or more of the plurality of steps in parallel without departing from the essential features of the present embodiment, so that the inventive concept is not limited to chronological order in FIGS. 6, 9, and 13.

The embodiments of the disclosure have been described above with reference to the accompanying drawings. A person skilled in the art to which this disclosure pertains will understand that the present disclosure may be practiced in forms different from the disclosed embodiments without changing the technical idea or essential features of the present disclosure. The disclosed embodiments are illustrative and should not be construed as limiting.

According to the aforementioned means to be resolved of the present disclosure, it is possible to increase the accuracy of high-frequency irradiation by accurately irradiating abundant electric energy or efficient electric energy during high-frequency irradiation.

According to the aforementioned means to be resolved of the present disclosure, it is possible to shorten a high-frequency irradiation time and maximize high-frequency irradiation effects.

According to the aforementioned means to be resolved of the present disclosure, it is possible to maximize optimal skin improvement effects of a monopolar type and a bipolar type while preventing skin burns in advance.

However, effects of the present disclosure may not be limited to the above-described effects. Although not described herein, other effects of the inventive concept can be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. An apparatus for applying monopolar and bipolar currents by skin depth during needle insertion, the apparatus comprising:
an electrode part including a plurality of needle-type electrodes inserted into skin;
an energy supply part configured to selectively apply a monopolar first current and a bipolar second current to the plurality of electrodes;
a transfer part configured to move the electrode part such that the plurality of electrodes reach a target depth in the skin; and
a processor configured to control the energy supply part such that the first current and the second current are applied to the plurality of electrodes at a current intensity corresponding to the reached target depth when the plurality of electrodes has reached the target depth,
wherein the processor is configured to:
perform control such that the first current and the second current are sequentially applied to the plurality of electrodes at a current intensity corresponding to a present first depth from a moment the electrode part is inserted into the skin, when the target depth is the first depth, and
perform control such that the second current and the first current are sequentially applied to the plurality of electrodes at a current intensity corresponding to a preset second depth from the moment the electrode part is inserted into the skin when the target depth is the second depth.

2. The apparatus of claim 1, wherein the processor is configured to perform control such that the first current and the second current are sequentially applied to the plurality of electrodes at the current intensity corresponding to the first depth until the electrode part is pulled out from the moment the electrode part is inserted into the skin when the target depth is the preset first depth.

3. The apparatus of claim 2, wherein the processor is configured to perform control such that there is a predetermined rest period between application times of the first current and the second current.

4. The apparatus of claim 3, wherein the processor is configured to perform control such that the second current and the first current are sequentially applied to the plurality of electrodes at the current intensity corresponding to the second depth until the electrode part is pulled out from the moment the electrode part is inserted into the skin when the target depth is the preset second depth.

5. The apparatus of claim 4, wherein the processor is configured to perform control such that there is a predetermined rest period between application times of the second current and the first current.

6. A method for being performed by an apparatus for applying monopolar and bipolar currents by skin depth during needle insertion, the method comprising:
moving, by a transfer part of the apparatus, an electrode part of the apparatus such that a plurality of electrodes reach a target depth in skin;
determining, by a processor of the apparatus, whether the plurality of electrodes has reached the target depth; and
controlling, by the processor of the apparatus, an energy supply part of the apparatus such that a monopolar first current and a bipolar second current are applied to the plurality of electrodes at a current intensity corresponding to the reached target depth when the plurality of electrodes has reached the target depth,
wherein the controlling includes:
performing control such that the first current and the second current are sequentially applied to the plurality of electrodes at a current intensity corresponding to a first depth from the moment the electrode part is inserted into the skin, when the target depth is the preset first depth; and
performing control such that the first current and the second current are sequentially applied to the plurality of electrodes at a current intensity corresponding to a second depth from the moment the electrode part is inserted into the skin, when the target depth is the preset second depth.

7. The method of claim 6, wherein the controlling includes performing control such that the first current and the second current are sequentially applied to the plurality of electrodes at the current intensity corresponding to the first depth until the electrode part is pulled out from the moment the electrode part is inserted into the skin when the target depth is the preset first depth.

8. The method of claim 7, wherein the controlling includes performing control such that there is a predetermined rest period between application times of the first current and the second current.

9. The method of claim 8, wherein the controlling includes performing control such that the second current and the first current are sequentially applied to the plurality of electrodes at the current intensity corresponding to the second depth until the electrode part is pulled out from the moment the electrode part is inserted into the skin when the target depth is the preset second depth.

10. The method of claim 9, wherein the controlling includes performing control such that there is a predetermined rest period between application times of the second current and the first current.
